(19) United States
(12) Patent Application Publication
(10) Pub. No.: US 2006/0134626 A1
(43) Pub. Date: Jun. 22, 2006

(54) Process for extracting nucleic acid

(76) Inventor: Hui Chen, Alhambra, CA (US)

Correspondence Address: SUTHERLAND ASBILL & BRENNAN LLP, 999 PEACHTREE STREET, N.E., ATLANTA, GA, 30309, US

(21) Appl. No.: 10/555,798

(22) PCT Filed: Mar. 5, 2004
(86) PCT No.: PCT/CN04/00177

§ 371 Date: Nov. 7, 2005

(30) Foreign Application Priority Data
May 6, 2003 (CN) 03122396.6

(51) Int. Cl. C12Q 1/68; C12N 1/08

(52) U.S. Cl. 435/6; 435/270

(57) ABSTRACT

The invention provides a process and kit for isolating and purifying nucleic acids such as DNA or RNA or a hybrid molecule of DNA and RNA. A siliceous material combined with a solution is used to prepare highly pure nucleic acids, especially DNA. The siliceous material is a support for absorbing target materials, and the solution according to this invention promotes the target materials to bind to the siliceous material, especially to promote DNA to bind to the siliceous material. The solution is an acid and potassium ion-containing aqueous solution. The invention further provides DNA prepared by using the process. Because no chaotropic agents or other poisonous or costly agents are used in the process, DNA prepared by the process may be used widely, especially in food industry and pharmaceutical industry.

### FIELD OF THE INVENTION

The present invention relates to the field of molecular biology. Particularly, the present invention relates to a unique solution and methods of use thereof for separating and purifying biological materials. More particularly, the present invention relates to a DNA purification method, in which silicon-containing materials are used to bind the target DNA in an acidic aqueous solution that contains potassium ions. The present invention provides highly purified DNA that is free from chaotropic agents or other poisonous agents and may be used widely, especially in food and pharmaceutical industries.

### BACKGROUND OF THE INVENTION

A method for separating and/or preparing highly purified target substances from different biomaterials is difficult because the natural biomaterials, such as tissue, cell, blood, bacteria, or the artificial biomaterials, such as the products of a polymerase chain reaction, are both complicated mixtures. However, such method is important because isolation and purification of target substances from the biomaterials are often needed in biomedical researches and/or other applications. For example, in a natural state, deoxyribonucleic acids (DNAs) are often mixed with other bio-substances, such as proteins, lipids and carbohydrates, isolating and purifying these DNA molecules containing a target gene are often necessary for further investigating the gene. Moreover, separating and purifying DNA, such as plasmid DNA, phage DNA and chromosome DNA are also important for researches in molecular biology, and for practical applications in pharmaceutical industry and gene therapy.

There are two kinds of DNA purification methods. One is the purification of artificially constructed DNAs. For instance, the purification of recombinant plasmids or phages from their cultivated hosts. This kind of DNA purification method is one of the basic techniques that is routinely used. The other DNA purification method is the purification of genomic DNAs from chromosomes of eukaryotes and prokaryotes. This kind of DNA purification method not only makes the researches on gene functions much easier but also makes the constructions of various DNA libraries available.

With the fast advancing researches in molecular biology and other related fields, there is a need for a new method for DNA isolation and purification that is safe, effective, and suitable for automation and industrialization. It has been reported that certain silicon-containing materials can absorb target substances in the presence of binding agents or binding enhancers. The target substances can then be purified by being eluted from the silicon carrier after the impurities are eliminated. U.S. Pat. No. 6,218,531 discloses a method for isolating RNAs from lysed biomaterials with silicon binding carrier in the presence of chaotropic reagents.

The underlying mechanism for these nucleic acid isolation and purification methods is that the silicon-containing materials can reversibly bind DNA, RNA and hybrid molecules of DNA and RNA in the presence of binding reagents. It has been reported that one of the most important binding reagents is the chaotropic reagent. Some common chaotropic reagents include NaI, urea, guanidine hydrochloride, NaClO4, and KBr. Alcohol, such as 100% ethanol, is also a commonly used binding reagent for nucleic acid purification (See the background of European Patent App. No. 0512676 A1 and U.S. Pat. No. 5,783,686). Because most of the binding reagents are toxic and harmful to human beings, reducing the use of toxic binding reagents or not using any toxic binding reagents are usually preferred. See U.S. Pat. No. 5,342,931 (filed on Apr. 23, 1993); U.S. Pat. No. 5,503,816 (filed on Nov. 17, 1994); U.S. Pat. No. 5,693,785; U.S. Pat. No. 5,674,997 (May 10, 1995).

U.S. Pat. No. 5,342,931 discloses a DNA purification method that uses hydroxylated silica polymers as a DNA binding carrier. There, the DNA was able to bind to the hydroxylated silica polymers in aqueous solutions or physiological buffers, and the bound DNA was eluted by hot water or physiological buffer after the bound DNA was isolated and the impurities were washed away.

The U.S. Pat. No. 5,503,816 discloses a method for chemically modified silicon-containing materials with a sufficient hydrophilicity and electropositivity. It has been noticed that the preferably modified siliceous materials are boron silicate, aluminum silicate, phosphosilicate, silica carbonyl, silica sulfonyl and silica phosphonyl. Some of these materials can absorb and/or bind DNA and the bound DNA can be recovered in water without using any chaotropic reagents. However, the preparation of these modified siliceous materials needs special chemical equipments.

In U.S. Pat. No. 5,693,785, methods and compositions for separating and purifying DNA are disclosed. The compositions disclosed therein were hydroxylated silica polymers produced by reacting silicon dioxide with an alkaline solution, followed by an acidification. The hydroxylated silica can bind DNA in water without any additional binding reagents, such as chaotropic reagents or alcohol. The bound DNA can be eluted by hot water or a physiological buffer.

In U.S. Pat. No. 5,674,997, a method for purification of DNA is disclosed. There, the DNA binds to silicon-containing materials, such as boron silicate, aluminum silicate, phosphosilicate, and silica phosphonyl, and the bound DNA was able to be eluted from these silicon-containing materials only with water.

Furthermore, U.S. Pat. Nos. 5,342,931, 5,503,816, 5,693,785 and 5,674,997 also disclose methods for purification of DNA by using a reduced amount or in the absence of any binding reagents. However, all of these methods need special chemical modifications for silicon-containing materials, provided that special chemical equipments are required for these chemical modifications.

It should be noticed that, while using silicon-containing materials as reversible absorbing materials for nucleic acids, and in particular, for DNA, use of any traditional binding reagents, especially chaotropic reagents, would be limited, if not avoided. Moreover, the use of chaotropic reagents or chaotropic salts is forbidden in the food and pharmaceutical industries because even a little trace residual of chaotropic reagents would be very harmful to human beings. Many efforts have been provided to solve the problems relating to nucleic acid purification in the absence of any binging reagents. However, the existing techniques focus on the improvements for special preparations and modifications of silicon-containing materials. Therefore, the current available techniques for nucleic acid purification have limitations for the applications of these methods and require extra efforts to study the characteristics of these specially modified siliceous materials. Moreover, the current available techniques are inconvenient to perform and quite expensive.

Therefore, there is a need to provide a new, effective and nontoxic binding reagent that can be used with the conventional silicon-containing materials for nucleic acid purification.

### SUMMARY OF THE INVENTION

In the first aspect of the present invention, an acidic aqueous solution containing potassium ions is provided. The acidic aqueous solution comprises: a) potassium ions, wherein the final concentration of the potassium ions in the acidic aqueous solution is in the range of 0.3 M to saturated; and b) acids to adjust the pH of the acidic aqueous solution in the range of 2.0-4.0.

The potassium ions in the acidic aqueous solution are derived from any potassium salts. Preferably, the potassium salts include but are not limited to: K₂SO₄, KNO₃, KCl, potassium acetate, or any mixture of the potassium salts thereof. No matter what kind of potassium salts are used, the final concentration of the potassium ions in the acidic aqueous solution is at least equal to or greater than 0.3M, and the pH of the acidic aqueous solution is in the range of 2.0-4.0, adjustable by any acids. The acids used for adjusting the pH of the acidic aqueous solution are weak acids or strong acids. In a preferred embodiment, weak acids, such as acetic acid, are used.

In the second aspect of the present invention, applications of the potassium ion-containing acidic aqueous solution are provided. In one of the preferred embodiments, the potassium ion-containing acidic aqueous solution is used for isolation and purification of biomaterials, such as nucleic acids including DNA, RNA or hybrids of DNA and RNA. For example, adding an appropriate amount of the potassium ion-containing acidic aqueous solution into a mixture of biomaterials containing target DNA enhances the binding of DNA to any of the conventional siliceous materials. After washing out the impurities that do not bind to the siliceous materials, the bound DNA can be separated and further purified from the siliceous materials. The solute including the potassium ions in the acidic aqueous solution can certainly enhance the binding of DNA to silicon-containing materials in the solution.

In the third aspect of the present invention, methods for purification and isolation of biomaterials are provided. Such methods comprise the steps of first combining an appropriate amount of the potassium ion-containing acidic solution with biomaterials that contain target bio-substances, such as DNA, RNA and/or the hybrids of DNA and RNA, and then combining the mixture with any of the conventional siliceous materials. Because the potassium ion-containing acidic solution of the present invention enhances the binding of the target bio-substances to any siliceous materials, the target bio-substances can be isolated and purified without adding any additional binding reagents in the mixture.

In the fourth aspect of the present invention, methods for purification and isolation of nucleic acids are provided. Such methods comprise the steps of first combining an appropriate amount of the potassium ion-containing acidic solution with biomaterials that contain target nucleic acids, and further combining the mixture with any of the conventional siliceous materials. Because the potassium ion-containing acidic solution enhances the binding of target nucleic acids to any siliceous materials, the target nucleic acids can be isolated and purified without adding any additional binding reagents in the mixture.

In the fifth aspect of the present invention, methods for purification and isolation of DNA are provided. Such methods comprise the steps of first combining an appropriate amount of the potassium ion-containing acidic solution with biomaterials that contain target DNA, and further combining the mixture with any of the conventional siliceous materials. Because the potassium ion-containing acidic solution enhances the binding of target DNA to any siliceous materials, the target DNA can be isolated and purified without adding any additional binding reagents in the mixture.

In the sixth aspect of the present invent, kits for nucleic acids, such as DNA, isolation and purification are provided. The kits comprise the potassium ion-containing acidic solution that comprises an appropriate amount of potassium salt or a mixture of potassium salts, an appropriate amount of an acid solution, and other necessary reagents for isolation and purification of DNA. The kits also comprise all of the reagents or the main reagents necessary for isolation and purification of DNA or other nucleic acids. The kits can further comprise an operating manual providing procedures for nucleic acid separation or purification.

In the seventh aspect of the present invention, the highly purified and the toxic reagent free DNA is provided using the method and the potassium ion-containing acidic solution provided by the present invention. The isolated DNA obtained herein is high pure and free from any poisonous agents. Such isolated and purified DNA can be widely used in a pharmaceutical industry, as well as in the food and cosmetic industries.

### DETAILED DESCRIPTION OF THE INVENTION

A binding carrier has a characteristic of reversible association and/or dissociation with a target substance. Such reversible association and/or dissociation characteristics of a binding carrier provide a basis of using such binding carrier for the separation of the target substance from a mixture. Silicon-containing materials are one of the binding carriers having such reversible association and/or dissociation characteristics and are widely used in the separation and purification of nucleic acids, such as DNA. In general, the binding of nucleic acids to siliceous materials, such as silica, celite, glass powders and the like, needs a presence of a high concentration of chaotropic reagents or alcohol. To avoid disadvantages associated with the use of chaotropic reagents and alcohol as binding reagents in the separation and purification of nucleic acids, several methods have been developed. One of the methods focuses on a chemical modification of siliceous materials, or provides special siliceous materials under special conditions. All of these methods increase the difficulties of the procedure for nucleic acid purification and limit the use of the binding carriers.

The present invention, however, provides a new nucleic acid binding solution for nucleic acid purification. The method for nucleic acid purification using the binding solution of the present invention not only avoids the use of any poisonous binding reagents but also enhances the binding of the nucleic acids to a variety of different silicon-containing materials. Therefore, the present invention provides a novel binding reagent that is nontoxic, inexpensive and easy to prepare. Such binding reagent can be a good suitable substitute for the traditional binding reagents, such as chaotropic reagents, in the course of nucleic acid extraction and purification.

In one of the preferred embodiments, the present invention provides a nucleic acid binding solution that is an acidic aqueous solution and contains potassium ions. The final concentration of the potassium ions in the acidic solution combined with any raw biomaterial is at least equal to or greater than 0.3 M, and the pH of the mixed solution is in the range of 2-4, adjusted by any acids. In a preferred embodiment, the acidic binding solution is an aqueous solution containing acetic acid and potassium chloride. The final concentration of the acidic acid in the mixed solution is in the range of 1-7 mol/L and the final concentration of potassium chloride in the mixed solution is in the range of 0.3 M to saturation. The potassium ions in the binding solution can be derived from K₂SO₄, KNO₃ and potassium acetate. A mixture of different potassium salts, such as the mixture of KCl and K₂SO₄, can also be used to provide potassium ions in the acidic aqueous binding solution. The acids used herein comprise weak acids and/or strong acids. Weak acids, such as acetic acid or propionic acid, are preferably used herein because it is more convenient to use weak acids to adjust the pH of the binding solution. Preferably, acetic acid is used to adjust the pH of the binding solution. The strong acids, such as hydrochloric acid, sulfuric acid, nitric acid and phosphoric acid, can also be used herein to adjust the pH of the binding solution.

The present invention provides a potassium ion-containing acidic aqueous binding solution and a method for nucleic acid purification using such binding solution. The present invention further provides that the final concentration of the potassium ions in the mixed binding solution is at least 0.3 M to saturation, and the pH of the mixed binding solution is in the range of 2-4. Because of the difference in the solubility of various potassium salts in water, the maximum concentration of the potassium salts in the present invention is the saturation. Since the solubility of potassium salts in a solution is changed with the temperature, in one of the preferred embodiments, the saturated concentration of a potassium salt is measured under room temperature. However, in yet another embodiment, the saturated concentration of a potassium salt can be measured under different temperatures other than room temperature. Because the saturated concentration of the potassium salts changes with temperatures, the changes of saturated concentration of potassium ions under different temperatures are within the scope of the present invention.

The potassium ions of the present invention are derived from dissolving one or more potassium salts in water or any other physiological buffers. Accordingly, the solutes in the binding solution of the present invention can be one potassium salt or the mixture of several potassium salts. The final concentration of the potassium ions in the mixed binding solution is the sum of potassium ions derived from all of the dissolved potassium salts in the solution. The final concentration of the potassium ions in the mixed binding solution is at least 0.3 mol/L or up to the saturated concentration. As stated above, the temperature for measuring the concentration of the potassium salts can be room temperature or any other temperatures higher than room temperature. The present invention provides that the saturated concentration of the potassium salts changes with different temperatures.

The present invention provides that any potassium salts, such as KCl, K₂SO₄, KNO₃ and potassium acetate or mixtures thereof, can be used as a source for potassium ions in the acidic binding solution. Preferably, high soluble potassium salts are used herein. In a preferred embodiment, potassium chloride is used for the source of potassium ions in the acidic binding solution of the present invention.

In yet another embodiment of the present invention, the potassium ion-containing binding solution is an acidic aqueous solution. As used herein, acids are used to adjust the acidity of the binding solution. The appropriate acids used for adjusting the acidity of the binding solution include but are not limited to acetic acid, sulfuric acid, nitric acid and phosphoric acid. Any other substances which can be used to adjust the pH of the potassium-contained binding solution are within the scope of the present invention. Both strong and weak acids can be used in the present invention, but weak acids are preferred. In a preferred embodiment, acetic acid is used in the present invention.

In a further embodiment of the present invention, the potassium ion-containing acidic binding solution comprises potassium chloride as a source for the potassium ions and acetic acid for adjusting the pH of the binding solution. The final concentration of potassium chloride in this binding solution in which acetic acid is used to adjust the pH, is 0.3-2.5 mol/L. The final concentration of acetic acid is 1-7 mol/L. Preferably, the final concentration of potassium chloride is 1.5-2.5 mol/L and the final concentration of acetic acid is 3-5 mol/L. Further preferably, the final concentration of potassium chloride is 2-2.5 mol/L and the final concentration of acetic acid is 34 mol/L.

The present invention further provides that any conventional siliceous materials can be used with the potassium ion-containing acidic binding solution of the present invention in the process of nucleic acid extraction and purification. The preferred siliceous materials include but are not limited to silica, glass celite, and the like. The siliceous materials used herein can be in different forms providing sufficient surfaces to absorb target biomaterials, such as DNA. For example, the silicon-containing materials of glass can be powder or fiber. The silicon-containing materials, such as glass powder, glass fiber or celite which exhibit sufficient hydrophilicity and electropositivity are preferable. The special silicon-containing materials disclosed in U.S. Pat. Nos. 5,342,931, 5,503,816, 5,693,785 and 5,674,997 can also be used in the present invention. In one of the preferred embodiments of the present invention, the silicon-containing material can be in the form of powder, which can be suspended in the potassium ion-containing acidic binding solution and be used to absorb the target biological substances, such as DNA. The siliceous materials can also be filled into a column for absorbing and/or binding to the target biomaterials and for further separation and purification of the bound target biomaterials thereafter.

The present invention further provides that the potassium ion-containing acidic binding solution enhances a binding of target biomaterials, especially DNA, to various silicon-containing materials. Accordingly, high efficiency of nucleic acid/DNA purification is achieved by the present invention. Furthermore, the highly purified DNA obtained by the present invention can be used in many fields, especially in the pharmaceutical industry. Based on the present invention, there is no limitation of using siliceous materials. Various siliceous materials including unmodified, modified and/or specially prepared silicon-containing materials can be used in the present invention. As used herein, the term “siliceous materials” and/or “silicon-containing materials” refers to any materials containing silicon, such as silica, glass celite, and hydroxylated silica, that are unmodified, modified, or specially treated and/or prepared under special conditions.

The present invention provides a method for separation and purification of biomaterials in which no specially treated or prepared silicon-containing materials and/or chaotropic reagents is required and/or used in the mixed solution. In one of the preferred embodiments of the present invention, the separation and purification method for extracting target bio-substances from a raw biomaterial comprises the following steps: (a) adding an appropriate amount of the potassium ion-containing acidic binding solution into a raw biomaterial mixture containing target bio-substances; (b) mixing the potassium ion-containing acidic binding solution with the raw biomaterial mixture thoroughly; (c) adding silicon-containing materials to the mixed binding solution to absorb the target biosubstances in the raw biomaterial; and (d) eluting and separating the target bio-substances from the bound silicon-containing materials. The method of the present invention also comprises a step of washing out the impurities of the raw biomaterials from the bound materials. As used herein, the target bio-substances can be proteins, nucleic acids, or any other bio-substances. As used herein, the nucleic acids refer to DNA, RNA and any hybrids of DNA and RNA molecules. As provided herein, the target bio-substances in the raw biomaterial mixture can be absorbed and bound specifically to the silicon-containing materials after adding an appropriate amount of the potassium ion-containing acidic binding solution of the present invention. Preferably, the present invention is used for separation and purification of DNA. The present invention also provides that silicon-containing materials can absorb DNA reversibly and selectively when the potassium ion-containing acidic binding solution of the present invention is added to the raw biomaterial mixture containing target DNA. In the present invention, an acidic binding solution containing potassium ions is preferably used. In yet another embodiment of the present invention, an adequate amount of the solute of the acidic binding solution of the present invention can also be used, if necessary. The present invention further provides that the silicon-containing materials can also absorb the target biomaterials, particularly DNA in the acidic binding solution of the present invention when the concentration of the potassium salts is saturated.

As used herein, the term “separation” refers to the isolation of target bio-substances from an original raw biomaterial mixture. For the separation of plasmid DNA, the term “separation” refers to a procedure to purify the plasmid DNA from cultured bacteria containing amplified plasmid DNA. The term “purification” refers to a procedure to purify the target bio-substances from impurities and/or to improve the purity of the target bio-substances. Because the terms “separation,” “purification,” “extraction,” “isolation,” and “preparation” of nucleic acids or other target bio-substances of the present invention involve the same core procedures, as used herein, these terms are used interchangeable in the present invention.

The present invention further provides a method for extraction of plasmid DNA using the potassium ion-containing acidic binding solution of the present invention. The method comprising the following steps: first, separating the bacteria from culture media and lysing the bacteria with NaOH-SDS using an alkaline lysis method; second, adding an appropriate amount of the potassium ion-containing acidic binding solution of the present invention and mixing it thoroughly with the lysate; third, adding a silicon-containing material into the mixed binding solution to absorb and bind the plasmid DNA; fourth, separating the silicon-containing material bound plasmid DNA and washing out the impurities; and finally, eluting the plasmid DNA from the silicon-containing material to obtain the purified plasmid DNA. Accordingly, the present invention provides an improvement for extraction of DNA by mixing an appropriate amount of the potassium ion-containing acidic binding solution of the present invention with the bacteria lysate that contains target DNA, and further with the silicon-containing material that absorbs and binds to the target DNA reversibly and selectively.

The present invention provides that the potassium ion-containing acidic binding solution of the present invention provides a condition for the target bio-substances, particularly DNA, to bind to the silicon-containing materials. Accordingly, the potassium ion-containing acidic binding solution of the present invention is preferably to be added into the starting raw biomaterial mixture before adding the silicon-containing materials for the DNA binding. The present invention further provides that the amount, such as the volume or the concentration, of the potassium ion-containing acidic binding solution that is added into the raw biomaterial mixture not only depends on the acidity and the concentration of the acidic binding solution itself but also depends on the acidity and the concentration of the potassium ions of the original raw biomaterial mixture. Accordingly, when the potassium ion-containing acidic binding solution of the present invention is used to separate and purify a particular target bio-substance, the concentration of the potassium ions in such acidic binding solution and the pH of such solution can be varied according to the concentration of the potassium ions in the raw biomaterial mixture and the pH of the raw biomaterial mixture. Such variations are within the scope of the present invention. More specifically, the technical index of the present invention in separating and purifying target bio-substances from a raw biomaterial are the pH and concentration of potassium ions in the raw biomaterial containing the target bio-substances, and the pH and the concentration of potassium ions in the final mixture solution after the potassium ion-containing acidic binding solution of the present invention is added into the raw biomaterial but before the binding of target bio-substances to the silicon-containing material. The binding condition of the present invention may also be achieved by adding the solute of the potassium ion-containing acidic binding solution of the present invention into the mixed binding solution. Therefore, the present invention provides a specific binding condition for a target bio-substance to bind to a silicon-containing material in the presence of at least 0.3 M to saturated potassium ions in an acidic environment. The present invention further provides a condition for separating and purifying target bio-substances in an acidic environment in the presence of at least 0.3M to saturated potassium ions in the mixed binding solution.

In further preferred embodiments, the present invention provides that, in order to separate and purify DNA from a raw biomaterial mixture and before adding the siliceous materials to absorb the DNA in the biomaterial mixture, the final concentration of the potassium ions in the mixture of the raw biomaterial with the acidic binding solution is at least equal to or greater than 0.3 mol/L and the pH of the mixture of raw biomaterial and the acidic binding solution is in the range of 2.0-4.0. The present invention also provides that the final concentration of the potassium ions in the mixture can be as high as saturated before any silicon-containing materials are added into the mixture to absorb and bind the DNA in the mixture. Accordingly, the maximum concentration of the potassium ions in the mixture before any silicon-containing materials are added is a saturated concentration. Alternatively, the solute of potassium salts can also be used to adjust the final concentration of the potassium ions in the mixture to reach at least 0.3M to saturated. In a preferred embodiment, the acidic aqueous solution containing potassium ions is used in the present invention.

Accordingly, in one aspect, the present invention provides a binding condition in which biological target substances can bind to silicon-containing materials, when the final concentration of the potassium ions in the binding condition is in the range of 0.3 mol/L to saturate and the pH of the binding condition is in the range of 2.0-4.0. In this binding condition, the silicon-containing materials can absorb target bio-substances, especially DNA.

Furthermore, the present invention includes a method for regulating the acidity and the concentration of the potassium ions in the target mixture before the target bio-substance in the mixture binds to the silicon-containing material. In a preferred embodiment, the regulation of the acidity and the concentration of the potassium ions in the target mixture is achieved by adding an appropriate amount of the acidic binding solution containing potassium ions into the target mixture. Alternatively, such a regulation step can be omitted if the acidity and the final potassium ion concentration in the target mixture is satisfied for the binding condition provided herein. Preferably, the present invention provides a special condition for the target bio-substances, especially DNA and RNA, to bind to the silicon-containing material in the absence of any additional binding agents, such as chaotropic agents, in the mixture. Alternatively, any methods providing the binding conditions as described herein that enable the target bio-substances to bind to any silicon-containing materials in the absence of any additional binding agents are within the scope of the present invention.

Therefore, the present invention provides a method of binding the target bio-substances, especially DNA, to any of the silicon-containing materials in a condition in which, before the silicon-containing materials are added in the mixed solution, the final concentration of the potassium ions in the mixed binding solution is at least 0.3 mol/L or more to saturate, and the pH of the mixed binding solution is in the range of 2.0-4.0.

In a preferred embodiment of the present invention, the plasmid DNA is separated from the bacteria of E. coli. The bacteria is separated from the culture media and lysed by NaOH-SDS. The concentration of the potassium ions in the lysate and the acidity of the lysate are regulated to the special binding condition as provided herein. The silicon-containing material is added to bind the plasmid DNA. The bound plasmid DNA is then separated by washing out the impurities, and the purified plasmid DNA is finally eluted from silicon-containing material using a conventional method known to the art.

In another embodiment of the present invention, the DNA is recovered from an aqueous solution. The DNA-containing mixture is adjusted to the binding condition provided by the present invention by adding an appropriate amount of the acidic binding solution containing potassium ions. A silicon-containing material is then added to bind the DNA and the bound DNA is recovered by a conventional method known in the art, for instance, centrifugation is provided to separate the bound DNA from impurities. After further washing out the impurities from the bound DNA, the pure DNA is eluted and achieved.

In yet another preferred embodiment, the method of the present invention can be repeated for providing DNA with even higher purity. For example, the biomaterial mixture containing target DNA is adjusted to the special binding condition as provided by the present invention by adding an appropriate amount of the acidic binding solution containing potassium ions. A silicon-containing material is then added to bind the DNA and the bound DNA is recovered by a conventional method known in the art, for instance, a centrifugation is provided to separate the bound DNA from the impurities. After washing out the impurities from the bound DNA, the eluted DNA can be applied to the same disclosed method again, and the DNA with an anticipated high purity can be obtained after several repeats of the method provided by the present invention.

Accordingly, the present invention provides a special binding solution for nucleic acid purification that is inexpensive, nontoxic to human beings, and easy to prepare. The binding solution provided by the present invention enhances the binding of a target biological material to a silicon-containing material without any special treatments for the silicon-containing material, and without any additional binding agents, such as chaotropic agents. The use of the binding solution of the present invention for DNA purification includes the procedures as provided above. In certain conditions, centrifuging the raw biomaterial mixture with the special binding solution of the present invention and obtaining the supernatant of the mixed solution for further binding of the target bio-substances to the silicon-containing material under the conditions provided by the present invention is necessary before adding the silicon-containing material into the mixed binding solution.

Accordingly, the present invention provides a method of separation and purification of nucleic acids, especially DNA and RNA, without using any additional toxic binding agents, such as chaotropic agents. The present invention provides that the silicon-containing material is used to absorb the target bio-substances, particularly DNA, in a mixed binding solution in which the final concentration of the potassium ions in the mixed binding solution is at least 0.3 mol//L or more to the saturate, and the pH of the mixed binding solution is in the range of 2-4. Moreover, the adjustment for the concentration of the potassium ions in the mixed binding solution and the adjustment for the acidity of the mixed binding solution is easy to perform based on the method of the present invention. Because the nucleic acids, especially DNA, are isolated and purified without using any toxic binding agents, the nucleic acids/DNA provided by the present invention meet the requirements for use of these products in pharmaceutical, food and cosmetic industries.

A wide range of common silicon-containing materials can be used in the present invention, because neither modified nor specialized silicon-containing material is needed. Generally the common silicon-containing materials are more stable than those of chemically modified materials. The chemical reagents used in the present invention are inexpensive, harmless to human beings and suitable for large scale preparation of DNA. Moreover, a high efficiency of nucleic acid separation and purification is provided by the present invention. High yields of nucleic acids/DNA can be achieved from the raw biomaterial using the method provided by the present invention.

The solute of the binding solution of the present invention can be provided as a mixture in a small package and can be further dissolved in water. It can also be provided with other supplement substances in a kit that can be used for DNA purification or other purposes. Any applications of using the binding solution of the present invention in an existing kit, or in a new kit are within the scope of the present invention. For example, the present invention provides a kit for preparation of plasmid DNA from E coli. Such kit comprises alkaline lysis reagent, the binding solution of the present invention, and other necessary reagents for plasmid DNA preparation. Any components in the kit can be provided in bottles or other containers. The kit of the present invention can also include an operating manual listing contents of the kit and providing procedures for DNA preparation or purification.

Furthermore, the binding solution and the binding condition of the present invention can be applied to any existing instruments known in the art for nucleic acid preparation and purification. Any of such applications of using the binding solution and the binding condition for nucleic acid preparation and purification are within the scope of the present invention.

Because DNA or other purified bio-substances prepared by the method of the present invention do not contain chaotropic agent residual which is prohibited in a pharmaceutical industry, the DNA and other purified bio-substances provided by the present invention can be widely used in many fields including biological experiments, pharmaceutical industry, food industry, cosmetic industry and nutritional industry.

### EXAMPLES

Throughout this application, various publications are referenced. The disclosures of all of these publications and those references cited within those publications in their entireties are hereby incorporated by reference into this application in order to more fully describe the state of the art to which this invention pertains.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### Example 1

### The Effect of pH on Separation and Purification of Plasmid DNA

Procedure—Extraction and Purification of Plasmid DNA from E. coli

1) 1.5 ml of over night culture of E. coli (HB101, containing pUC19) was transferred to a 1.5 ml centrifuge tube (totally 7 tubes, marked No. 1-7), and centrifuged for 30 sec at 12000 g. The supernatant was discarded;

2) 200 μl of Solution A was added to the precipitated bacteria pellet and re-suspended thoroughly;

3) 200 μl of solution B was added and mixed thoroughly with (2) by inverting the tube up and down. The mixture was stood at room temperature for 3 min;

4) 250 μl of solution C was added and mixed thoroughly with (3) by inverting the tube up and down. The mixture was then centrifuged at 15000 g for 10 min at 4° C.;

5) The supernatant was carefully transferred to a column in which 50 mg glass powder or glass fiber was added. The column with the transferred supernatant was inserted into a 2 ml collecting tube and centrifuged at 15000 g for 1 min. The flow through in the collection tube was discarded;

6) 450 μl of solution D was then added into the column, and the column with solution D was centrifuged at 15000 g for 30 sec. The flow through in the collection tube was discarded;

7) another 450 μl of solution D was added into the column, and the column was further centrifuged at 15000 g for 2 min;

8) The column was then carefully transferred into a clean 1.5 ml centrifuge tube and 100 μl of solution E was added to the column. After 1-2 min standing at room temperature, the column was then centrifuged at 15000 g for 1 min, the plasmid DNA was eluted in solution E.

Solutions: As used herein:

Solution A contains 20 μg/ml RNase A, 50 mM Tris-HCl (pH8.0), and 10 mM EDTA (pH8.0).

Solution B contains 0.2 M NaOH, and 1% SDS

Solution C contains 2.5M KC and acetic acid (HAC). Various HAC concentrations in solution C are shown in table 1.

Solution D is 70% ethanol, and

Solution E contains 10 mM Tris-HCl and 5 mM EDTA, pH 8.0.

TABLE 1

| | HAC | KCl | System pH | |
|---|---|---|---|---|
| | concentration | concentration | after adding | DNA |
| Sample | in solution C | in solution C | of solution | yield |
| No | (M) | (M) | A, B, C | (μg) |
| 1 | 1 | 2.5 | 3.9 | 10.0 |
| 2 | 2 | 2.5 | 3.6 | 15.9 |
| 3 | 3 | 2.5 | 3.3 | 27.1 |
| 4 | 4 | 2.5 | 3.1 | 33.8 |
| 5 | 5 | 2.5 | 3.0 | 29.1 |
| 6 | 6 | 2.5 | 2.8 | 25.3 |
| 7 | 7 | 2.5 | 2.6 | 20.2 |

Note:
the pH of the system after mixing solutions A, B, and C is the pH of the mixed solution containing target substance after adding the solutions of the present invention and before the binding of target substance to silicon-containing materials.

Table 1 shows the effect of changing the concentration of acetic acid in solution C leading to the changes of the pH of the mixed binding solution before the binding of target substance to silicon-containing materials occurred, whereas the concentration of the potassium ions in the mixed binding solution kept constant. It is noticed that changes of the pH of the mixed binding solution remarkably affect the yield of DNA. The present results showed that a higher yield of DNA was achieved when the concentration of the potassium ions in the mixed binding solution was 2.5M and the concentration of acetic acid in the mixed binding solution was 4M, provided the pH of the mixed binding solution was 3.1 before the binding of DNA to silicon-containing materials occurred.

### Example 2

### Effect of Potassium Ion Concentration on Separation and Purification of Plasmid DNA

Procedure—Extraction and Purification of Plasmid DNA from E. Coli

1) 1.5 ml of over night culture of E. coli (HB101, containing pUC19) was transferred to a 1.5 ml centrifuge tube (totally 7 tubes, marked No. 1-7), and centrifuged for 30 sec at 12000 g. The supernatant was discarded;

2) 200 μl of solution A was added to the precipitated bacteria pellet and re-suspended thoroughly;

3) 200 μl of solution B was added and; mixed thoroughly with (2) by inverting the tube up and down. The mixture was stood at room temperature for 3 min;

4) 250 μl of solution C was added and; mixed it thoroughly with (3) by inverting the tube up and down. The mixture was then centrifuged at 15000 g for 10 min at 4° C.;

5) The supernatant was carefully transferred to a column in which 50 mg glass powder or glass fiber was added. The column with the transferred supernatant was inserted into a 2 ml collecting tube and centrifuged at 15000 g for 1 min. The flow through in the collection tube was discarded;

6) 450 μl of solution D was added into the column and the column was centrifuged at 15000 g for 30 sec. The flow through in the collection tube was discarded;

7) another 450 μl of solution D was added into the column, and the column was further centrifuged at 15000 g for 2 min;

8) The column was then carefully transferred into a clean 1.5 ml centrifuge tube and 100 μl of solution E was added to the column. After 1-2 min standing at room temperature, the column was then centrifuged at 15000 g for 1 min, and the plasmid DNA was eluted in solution E.

Solutions—As used herein, solutions A, B, and E are the same as those in Example 1. Solution C, on the other hand, contains 2M HAC, and different concentration of KCl. Various concentrations of KCl in solution C were presented in Table 2.

TABLE 2

| | System pH | | |
|---|---|---|---|
| | after adding | System KCl concentration | DNA |
| Sample | of solution | after adding of | yield |
| No | A, B, C | solution A, B, C (M) | (μg) |
| 1 | 3.1 | 0.25 | 9.0 |
| 2 | 3.1 | 0.50 | 13.2 |
| 3 | 3.1 | 0.75 | 22.3 |
| 4 | 3.1 | 1.00 | 32.1 |
| 5 | 3.1 | 1.25 | 33.4 |
| 6* | 3.1 | 1.75 | 32.7 |
| 7* | 3.1 | 2.5 | 33.1 |

*Solid potassium chloride power was add to the solutions A, B, and C mixing system to increase the KCl concentration to 1.75M and 2.5M, respectively in 6 and 7.

Table 2 shows the effect of changing the concentration of the potassium ions in solution C providing the changes of the potassium ions in the mixed binding solution before the binding of DNA to silicon-containing materials occurred, whereas the pH of the mixed binding solution kept constant. It is noticed that changes of the concentration of the potassium ions in the mixed binding solution significantly affects the yield of DNA. The present results also showed that the high DNA yield was achieved when the pH of the mixed binding solution was 3.1 and the concentration of the potassium ions in the mixed binding solution was 1M or higher. The yield of DNA did not increase significantly when the concentration of potassium ions is more than 1M in the mixed binding solution. Accordingly, the preferable concentration of the potassium ions in the mixed binding solution is 1M, and the concentration of the potassium ions in solution C could be easily calculated from the concentration of the potassium ions in the mixed solutions of A, B, and C.

### Example 3

### Effect of Different Kinds of Potassium Salts on Separation and Purification of Plasmid DNA

Procedure—Extraction and Purification of Plasmid DNA from E. Coli

1) 1.5 ml of over night culture of E. coli (HB101, containing pUC19) was transferred to a 1.5 ml centrifuge tube (totally 3 tubes, marked No. 1-3), and centrifuged for 30 sec at 12000 g. The supernatant was discarded;

2) 200 μl of solution A was added to the precipitated bacteria pellet and re-suspended thoroughly;

3) 200 μl of solution B was added and mixed thoroughly with (2) by inverting the tube up and down. The mixture was stood at room temperature for 3 min;

4) 500 μl of solutions C₁, C₂, C₃ were added, respectively, and mixed thoroughly with (3) by inverting the tube up and down. The mixture was then centrifuged at 15000 g for 10 min at 4° C.;

5) The supernatant was carefully transferred to a column in which 50 mg glass powder or glass fiber was added. The column with the transferred supernatant was inserted into a 2 ml collecting tube and centrifuged at 15000 g for 1 min. The flow through in the collection tube was discarded;

6) 450 μl of solution D was added into the column, and the column was centrifuged at 15000 g for 30 sec. The flow through in the collection tube was discarded;

7) another 450 μl of solution D was added into the column, and the column was further centrifuged at 15000 g for 2 min. The flow through in the collection tube was discarded; and

8) The column was then carefully transferred into a clean 1.5 ml centrifuge tube and 100 μl of solution E was added to the column. After 1-2 min standing at room temperature, the column was then centrifuged at 15000 g for 1 min. The plasmid DNA was eluted in solution E from the column.

Solutions—As used herein, solutions A, B, D and E are the same as those of Example 1. Solutions C1, C2, and C3 comprise HAC 2M and K₂SO₄ (C1), KNO₃ (C2), and KCl (C3). The concentration of K₂SO₄, KNO₃, and KCl was listed in Table 3.

TABLE 3

| Solution C | | | | |
|---|---|---|---|---|
| | | Potassium | K+ | DNA yield |
| No. | HAC | salt | concentration(M) | (μg) |
| C1 | HAC 2M | K2SO4 | 1.4 | 21.5 |
| C2 | HAC 2M | KNO3 | 1.4 | 22.4 |
| C3 | HAC 2M | KCl | 1.4 | 20.9 |

The results presented in Table 3 showed that the changes of different kinds of potassium salts used in the solution C and the other ions from the resolved potassium salts did not affect the yield of DNA. Therefore, the final concentration of the potassium ions in the mixed binding solution, but not its source salts, is the important factor of the present invention.

### Example 4

### Recovery of DNA from an Aqueous Solution

Procedure:

1) 200 μl aqueous solution containing 7 μg DNA was transferred;

2) 100 μl of solution C was added and mixed with (1) by inverting the tube up and down;

3) The mixed solution of (1) and (2) was transferred to a column in which 50 mg glass powder or glass fiber was added. The column with the transferred mixed solution was inserted into a 2 ml collecting tube and centrifuged at 15000 g for 1 min. The flow through in the collection tube was discarded;

4) 450 μl of solution D was added into the column, and the column was then centrifuged at 15000 g for 30 sec. The flow through in the collection tube was discarded;

5) another 450 μl of solution D was added into the column, and the column was further centrifuged at 15000 g for 2 min;

6) The column was carefully transferred into a clean 1.5 ml centrifuge tube and 100 μl of solution E was added to the column. After 1-2 min standing at room temperature, the column was then centrifuged at 15000 g for 1 min. the DNA was eluted in solution E from the aqueous solution; and

7) The yield of DNA that eluted from the column is 5.1 μg measured at 260 nm.

Solutions—As used herein, the solutions D and E are the same as those of Example 1. Solution C contains 1M HAC and 2.5M KCl. Although the two components of solution C was not optimized (see example 1 and table 1), the yield of DNA was recovered up to 73% in this example. This result shows that the DNA in a raw material can be recovered well by using the extraction and purification method of the present invention. The present example not only shows the advantage of using the method of the present invention for extracting a trace amount of DNA in a mixture but also shows a little loss in DNA extraction by using the method of the present invention.

### Example 5

### The Effect of Different Acids Used for Adjusting the Acidity of the Potassium Ion-Containing Solution on Separation and Purification of Plasmid DNA

Procedure—Extraction and purification of plasmid DNA from E. coli

1) 1.5 ml of over night culture of E. coli (HB101, containing pUC19) was transferred to a 1.5 ml centrifuge tube (totally 4 tubes, marked No. 1-4), and centrifuged for 30 sec at 12000 g. The supernatant was discarded;

2) 200 μl of solution A was added to the precipitated bacteria pellet and re-suspended thoroughly;

3) 200 μl of solution B was added and mixed thoroughly with (2) by inverting the tube up and down. The mixture was stood at room temperature for 3 min;

4) 500 μl of solutions C₁, C₂, C₃, C₄ were added, respectively, to make the pH of the mixed solution of A, B and C be 3.1. The mixed solution of A, B and C was mixed thoroughly by inverting the tube up and down, and centrifuged at 15000 g for 10 min at 4° C.;

5) The supernatant was carefully transferred to a column in which 50 mg glass powder or glass fiber was added. The column with the transferred supernatant was inserted into a 2 ml collecting tube and centrifuged at 15000 g for 1 min;

6) 450 μl of solution D was added into the column, and the column was centrifuged at 15000 g for 30 sec. The flow through in the collection tube was discarded;

7) another 450 μl of solution D was added into the column, and the column was further centrifuged at 15000 g for 2 min; and

8) The column was then carefully transferred into a clean 1.5 ml centrifuge tube and 100 μl of solution E was added to the column. After 1-2 min standing at room temperature, the column was then centrifuged at 15000 g for 1 min. The plasmid DNA was eluted in solution from the column.

Solutions—As used herein, the solutions A, B, D, and E are as the same as those of Example 1. Solution C1 contains 0.2M HCl and 2.5M KCl. Solution C2 contains 0.2M HNO₃ and 2.5M KCl. Solution C3 contains 0.1M H₂SO₄ and 2.5M KCl. Solution C4 contains 4M HAC and 2.5M KCl (Table 4).

TABLE 4

| Solution C | Acid used | DNA Yield(μg) |
|---|---|---|
| C1 | HCl | 26.5 |
| C2 | HNO3 | 28.1 |
| C3 | H2SO4 | 24.9 |
| C4 | HAC | 26.9 |

The result of Example 5 showed that the function of an acid was to keep the mixed binding solution at an appropriate acidity, and the kind of acids used in the present invention did not significantly affect the yield of DNA extracted using the method of the present invention. Table 4 presents a similar DNA yield when the strong acids such as HCl, HNO₃, and H₂SO₄ were used. The yields of DNA were very close when the concentration of the H⁺ which was provided by different acids, was kept in the same level. Therefore, the function of the acid in the potassium ion-containing binding solution of the present invention is to provide H⁺ which keeps the solution in an appropriate acidity before the DNA binding to the silicon-containing materials occurs.

### INDUSTRIAL APPLICABILITY

The present invention provides a method of isolating biomaterials from other materials, particularly nucleic acids. The present invention also provides a method for purifying nucleic acids, particularly DNA from biomaterials. A silicon-containing material combining with a novel potassium ion-containing acidic solution is used in the method for preparing high purity biomaterials, particularly DNA. The present invention also provides a kit using the method of the present invention. The kit contains silicon-containing materials, the potassium ion-containing acidic solution and other needed reagents and solutions. Because no chaotropic reagent and any other toxic, expensive chemicals are used in the preparation of DNA by the method of the present invention, the DNA obtained by the present invention can be widely used, particularly in the pharmacological and food industries.

1. (canceled)

2. (canceled)

3. (canceled)

4. (canceled)

5. (canceled)

6. (canceled)

7. (canceled)

8. (canceled)

9. (canceled)

10. (canceled)

11. A method for separation and purification of nucleic acids, said method comprising:
(a) adding an appropriate amount of acidic aqueous solution containing potassium ions into a raw biomaterial containing nucleic acids;
(b) mixing said acidic aqueous solution containing potassium ions with said raw biomaterial, wherein a concentration of said potassium ions in a mixed binding solution is in a range of 0.3M to saturated, and a pH of the mixed binding solution is in a range of 2.0-4.0; wherein said potassium ions are selected from the group consisting of K₂SO₄, KNO₃, KCl, potassium acetate and a mixture thereof; and wherein said acidic aqueous solution is selected from the group consisting of acetic acid, propionic acid, hydrochloric acid, sulfuric acid, nitric acid, phosphoric acid and a mixture thereof;
(c) adding a silicon-containing material to said mixed binding solution, wherein said nucleic acids in said raw biomaterial are capable of binding to said silicon-containing material;
(d) separating nucleic acids bound with said silicon-containing material from impurities; and
(e) separating and eluting nucleic acids from said silicon-containing material.

12. The method of claim 11, wherein said nucleic acid is DNA.

13. The method of claim 11, wherein said concentration of potassium ions in said mixed binding solution is equal to or greater than 1M.

14. The method of claim 11, wherein said pH of said mixed binding solution is about 2.6-3.9.

15. A kit used for a DNA separation and purification, said kit comprising:
(a) an acidic aqueous solution containing potassium ions, wherein said acidic aqueous solution is selected from the group consisting of acetic acid, propionic acid, hydrochloric acid, sulfuric acid, nitric acid, phosphoric acid and a mixture thereof; wherein said potassium ions are selected from the group consisting of K₂SO₄, KNO₃, KCl, potassium acetate and a mixture thereof; and wherein a concentration of said potassium ions in a mixed binding solution is adjusted to be in a range of 0.3M to saturated, and a pH of the mixed binding solution is adjusted to be in a range of 2.0-4.0;
(b) a silicon-containing material that is capable of binding to said DNA; and
(c) an introduction manual listing contents of said kit and providing procedures for said DNA separation or purification.

16. A method of extracting plasmid DNA from cultured bacteria, said method comprising:
(a) providing a bacteria lysate by separating and lysing the bacteria from cultured media;
(b) providing a mixed binding solution by thoroughly mixing an appropriate amount of acidic aqueous solution containing potassium ions with the bacteria lysate, wherein a concentration of said potassium ions in said mixed binding solution is in a range of 0.3M to saturated, and a pH of the mixed binding solution is in a range of 2.0-4.0; wherein said potassium ions are selected from the group consisting of K₂SO₄, KNO₃, KCl, potassium acetate and a mixture thereof; and wherein said acidic aqueous solution is selected from the group consisting of acetic acid, propionic acid, hydrochloric acid, sulfuric acid, nitric acid, phosphoric acid and a mixture thereof;
(c) adding a silicon-containing material to said mixed binding solution, wherein said plasmid DNA is capable of binding to said silicon-containing material;
(d) separating said plasmid DNA bound with said silicon-containing material from impurities; and
(e) washing out the impurities and eluting said plasmid DNA from said silicon-containing material to obtain the purified plasmid DNA.

17. The method of claim 16, wherein said cultured bacteria is the bacteria of E. coli.

18. The method of claim 16, wherein said concentration of potassium ions in said mixed binding solution is equal to or greater than 1M.

19. The method of claim 16, wherein said pH of said mixed binding solution is about 2.6-3.9.

20. A kit used for extracting plasmid DNA from cultured bacteria, said kit comprising:
(a) reagents or solutions used for separating and lysing said cultured bacteria;
(b) an acidic aqueous solution containing potassium ions, wherein said acidic aqueous solution is selected from the group consisting of acetic acid, propionic acid, hydrochloric acid, sulfuric acid, nitric acid, phosphoric acid and a mixture thereof; wherein said potassium ions are selected from the group consisting of K₂SO₄, KNO₃, KCl, potassium acetate and a mixture thereof; and wherein a concentration of said potassium ions in a mixed binding solution of bacterial lysate and said acidic aqueous solution is adjusted to be in a range of 0.3M to saturated, and a pH of said mixed binding solution is adjusted to be in a range of 2.0-4.0;
(c) a silicon-containing material that is capable of binding to said plasmid DNA;
(d) reagents or solutions for separating and eluting bound plasmid DNA from impurities and said silicon-containing material; and
(e) an introduction manual listing contents of said kit and providing procedures for extracting said plasmid DNA.

21. An isolated endogenous nucleic acid free of chaotropic reagent and other toxic reagents, said isolated endogenous nucleic acid is obtainable by a method comprising:
(a) adding an appropriate amount of acidic aqueous solution containing potassium ions into a raw biomaterial containing said endogenous nucleic acid;
(b) providing a mixed binding solution by thoroughly mixing said acidic aqueous solution containing potassium ions with said raw biomaterial containing said endogenous nucleic acid, wherein a concentration of said potassium ions in said mixed binding solution is in a range of 0.3M to saturated, and a pH of said mixed binding solution is in a range of 2.0-4.0; wherein said potassium ions are selected from the group consisting of K₂SO₄, KNO₃, KCl, potassium acetate and a mixture thereof; and wherein said acidic aqueous solution is selected from the group consisting of acetic acid, propionic acid, hydrochloric acid, sulfuric acid, nitric acid, phosphoric acid and a mixture thereof;
(c) adding a silicon-containing material to said mixed binding solution, wherein said endogenous nucleic acid in said raw biomaterial are capable of binding to said silicon-containing material;
(d) separating silicon-containing material bound endogenous nucleic acid from impurities; and
(e) separating and eluting said endogenous nucleic acid from said silicon-containing material to obtain said isolated endogenous nucleic acid.

22. The isolated endogenous nucleic acid of claim 21, wherein said endogenous nucleic acid is endogenous DNA.

23. The isolated endogenous nucleic acid of claim 22, wherein said DNA is plasmid DNA.

24. The isolated endogenous nucleic acid of claim 21, wherein said concentration of potassium ions in said mixed binding solution is equal to or greater than 1M.

25. The isolated endogenous nucleic acid of claim 21, wherein said pH of said mixed binding solution is about 2.6-3.9.

26. An isolated endogenous nucleic acid free of chaotropic reagent and other toxic reagents.

27. The isolated endogenous nucleic acid of claim 26, wherein said isolated endogenous nucleic acid is DNA.

28. The isolated endogenous nucleic acid of claim 27, wherein said DNA is plasmid DNA.
